(19) United States
(12) Patent Application Publication
(10) Pub. No.: US 2022/0227769 A1
(43) Pub. Date: Jul. 21, 2022

(54) CRYSTALLINE FORM OF TELMAPITANT OR (5R,8S)-8-[[(1R)-1-[3,5-BIS(TRIFLUOROMETHYL)PHENYL]ETHOXY]METHYL]-8-PHENYL-1,3,7-TRIAZASPIRO[4.5]DECANE-2,4-DIONE

(71) Applicant: Intervet Inc., Madison, NJ (US)

(72) Inventors: Rositza Iordanova Petrova, Metuchen, NJ (US); Gregory William York, Lebanon, NJ (US); Arush Pandya, Cranford, NJ (US)

(73) Assignee: Intervet Inc., Madison, NJ (US)

(21) Appl. No.: 17/628,419

(22) PCT Filed: Jul. 24, 2020
(86) PCT No.: PCT/EP2020/070930

§ 371 Date: Jan. 19, 2022

Related U.S. Application Data
(60) Provisional application No. 62/878,530, filed on Jul. 25, 2019.

(51) Int. Cl. C07D 471/10

(52) U.S. Cl. CPC C07D 471/10; C07B 2200/13

(57) ABSTRACT

The present disclosure encompasses a crystalline form of (5R,8S)-8-[[(1R)-1-[3,5bis(trifluoromethyl)phenyl]ethoxy]methyl]-8-phenyl-1,3,7-triazaspiro[4.5]decane-2,4-dione and processes for the preparation thereof.

### BACKGROUND

U.S. Pat. No. 7,049,320 discloses compounds of Formula I which are an NK₁ antagonists and useful in the treatment of delayed onset emesis such as experienced one to several days after receiving chemotherapy. U.S. Pat. No. 7,049,320 discloses that compounds of Formula I can be in liquid form preparations including solutions, suspensions and emulsions.

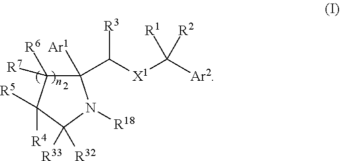

The compound telmapitant or (5R,8S)-8-[[(1R)-1-[3,5-bis(trifluoromethyl)phenyl]ethoxy]methyl]-8-phenyl-1,3,7-triazaspiro[4.5]decane-2,4-dione, CAS #552292-58-7, is also disclosed in U.S. Pat. No. 7,049,320.

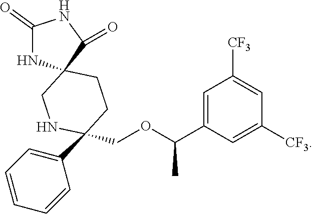 [SMILES: C[C@@H](OC[C@@]1(c2ccccc2)CC[C@]2(CN1)NC(=O)NC2=O)c1cc(C(F)(F)F)cc(C(F)(F)F)c1]

The tachykinin NK-1 receptor is part of a family of receptors that also includes the NK-2 and NK-3 receptors (L Quartara and C A Maggi, 1997, The tachykinin NK₁ receptor. Part I: ligands and mechanisms of cellular activation. Neuropeptides 31(6), 537-563).

The natural and most potent agonist for the NK-1 receptor is the tachykinin substance P. In the CNS, NK-1 receptors have been shown to be involved in behavioral responses, regulation of cardiovascular and respiratory function, and activating the emetic reflex. NK-1 antagonists have proven to be very effective antiemetics with distinct advantages over other classes of antiemetics. NK-1 antagonists have achieved regulatory approval for an antiemetic indication in both humans (aprepitant, i.e. Emend® and rolapitant, i.e. Varubi®), and in dogs (maropitant, i.e. Cerenia®). In dogs, maropitant had been shown to be effective against both centrally acting emetogens (apomorphine IV) and peripherally acting emetogens (syrup of ipecac orally). (See H S Sedlecek, et. al. 2008, J. Vet. Pharmacol. Therap. 31(6) 533-537).

NK-1 antagonists are also effective in treating postsurgical/post anesthesia-induced emesis, motion induced emesis, and emesis from disease (D S Ramsey, et. al. 2008, Safety and efficacy of injectable and oral maropitant, a selective neurokinin₁ receptor antagonist, in a randomized clinical trial or treatment of vomiting in dogs. J. Vet. Pharmacol. Therap. 31(6) 538-543).

None of the above references disclose the inventive polymorphic form of the compound.

### BRIEF DESCRIPTION OF THE DRAWINGS

FIG. 1 is a characteristic X-ray powder diffraction pattern of the crystalline Form 1.

FIG. 2 is a carbon-13 cross-polarization magic-angle spinning (CPMAS) nuclear magnetic resonance (NMR) spectrum of the crystalline Form 1.

FIG. 3 is a typical DSC curve of the crystalline Form 1.

FIG. 4 shows an overlay of the X-ray powder diffraction patterns of MK-7035 Form 1, Form 2, and Form 3.

FIG. 5 is a typical DSC curve of the crystalline Form 2.

FIG. 6 is a typical DSC curve of the crystalline Form 3.

### SUMMARY OF THE INVENTION

A crystalline form of (5R,8S)-8-[[(1R)-1-[3,5bis(trifluoromethyl)phenyl]ethoxy]methyl]-8-phenyl-1,3,7-triazaspiro[4.5]decane-2,4-dione having at least one of the following characteristics:
an X-ray power diffraction (XRPD) spectrum having at least one peak selected from the group consisting of 3.9 (±0.2), 17.2 (±0.2), and 27.8 (±0.2) degrees 2Θ;
a carbon-13 cross polarization magic-angle spinning (CPMAS) nuclear magnetic resonance (NMR) spectrum having at least one peak selected from the group consisting of 181.08, 158.94, 145.40, 141.48, 132.60, 131.72, 129.96, 128.58, 126.36, 122.68, 82.09, 81.66, 80.30, 62.96, 60.49, 50.62, 26.75, 24.97, 24.37, 22.60, and 21.65 ppm; or
a differential scanning calorimetry (DSC) thermogram comprising an endothermic peak at about 206° C.

### DETAILED DESCRIPTION

Three anhydrous polymorphic forms of telmapitant or (5R,8S)-8-[[(1R)-1-[3,5-bis(trifluoromethyl)phenyl]ethoxy]methyl]-8-phenyl-1,3,7-triazaspiro[4.5]decane-2,4-dione (MK-7035) have been identified during polymorph screening. Competitive slurry experiments of Form 1 with Form 2 and Form 3 in acetonitrile at room temperature (20-25° C.) and 75° C. showed that Form 1 is thermodynamically more stable than Form 2 and Form 3.

The crystalline anhydrous Form 1 of MK-7035 was characterized by X-ray powder diffraction (XRPD), carbon-13 solid state NMR (ssNMR), and Differential Scanning calorimetry (DSC).

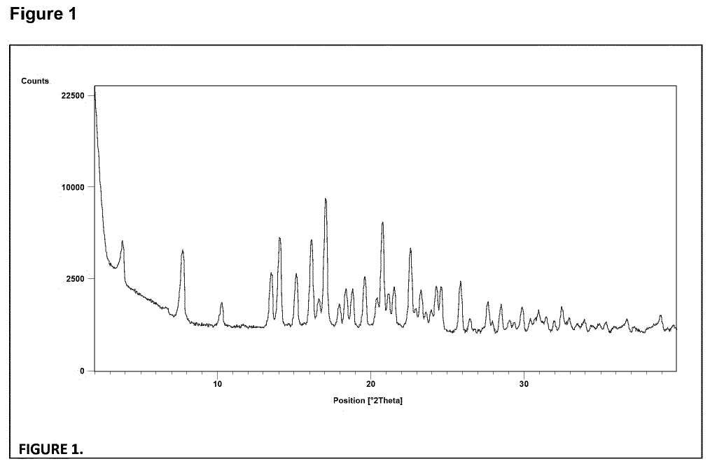

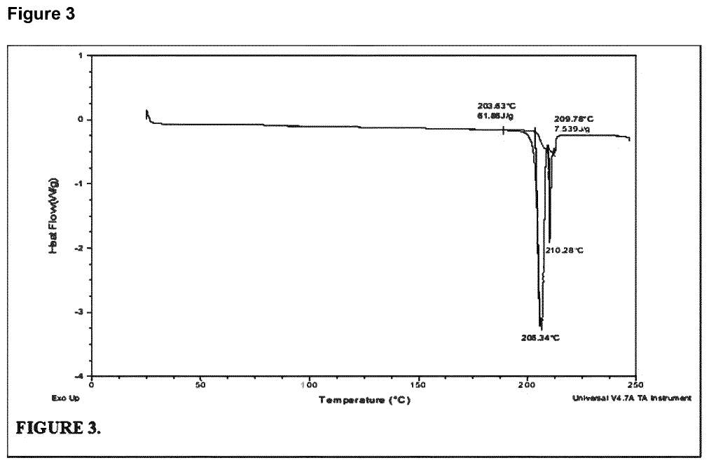

The term “substantially as shown” as used herein refers to an X-ray powder diffraction (XRPD) spectrum, carbon-13 cross-polarization magic-angle spinning (CPMAS) nuclear magnetic resonance (NMR) spectrum, or differential scanning calorimetry (DSC) thermogram that is non-identical to those depicted herein, but may fall within the limits of experimental error, when considered by one of ordinary skill in the art. One of ordinary skill in the art would understand that an X-ray powder diffraction spectrum may contain peaks that fall within ±0.2 degrees 2Θ of the peaks contained in the spectrum of FIG. 1 and a differential scanning calorimetry (DSC) thermogram as depicted in FIG. 3 may contain an endotherm at ±3° C. of what is depicted.

The term “substantially purified” as used herein refers to a crystalline form of the compound that is at least 90% Form 1 of (5R,8S)-8-[[(1R)-1-[3,5-bis(trifluoromethyl)phenyl]ethoxy]methyl]-8-phenyl-1,3,7-triazaspiro[4.5]decane-2,4-dione. In an alternate embodiment, “substantially purified” refers to a crystalline form of the compound that is at least 95%, 99%, or 99.9% Form 1 of (5R,8S)-8-[[(1R)-1-[3,5-bis(trifluoromethyl)phenyl]ethoxy]methyl]-8-phenyl-1,3,7-triazaspiro[4.5]decane-2,4-dione.

In an embodiment, the crystalline form of Form 1, having an X-ray powder diffraction (XRPD) spectrum substantially as shown in FIG. 1.

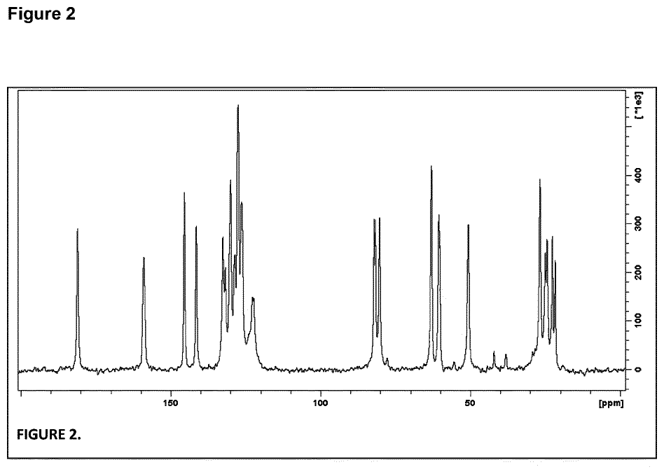

In an embodiment, the crystalline form of Form 1, having carbon-13 cross-polarization magic-angle spinning (CPMAS) nuclear magnetic resonance (NMR) spectrum substantially as shown in FIG. 2.

In an embodiment, the crystalline form of Form 1, having a differential scanning calorimetry (DSC) thermogram substantially as shown in FIG. 3.

In an embodiment, the crystalline for=—of (5R,8S)-8-[[(1R)-1-[3,5bis(trifluoromethyl)phenyl]ethoxy]methyl]-8-phenyl-1,3,7-triazaspiro[4.5]decane-2,4-dione having a carbon-13 cross-polarization magic-angle spinning (CPMAS) nuclear magnetic resonance (NMR) spectrum having at least one peak, at least two peaks, at least three peaks, at least four peaks, at least five peaks, at least six peaks, at least seven peaks, at least eight peaks, at least nine peaks, or at least ten peaks selected from the group consisting of 181.08, 158.94, 145.40, 141.48, 132.60, 131.72, 129.96, 128.58, 126.36, 122.68, 82.09, 81.66, 80.30, 62.96, 60.49, 50.62, 26.75, 24.97, 24.37, 22.60, and 21.65 ppm.

In an embodiment, the crystalline form of Form 1, wherein the crystalline form is thermodynamically stable at temperatures below about 75° C.

In an additional embodiment, a pharmaceutical composition comprising the crystalline form of Form 1 and a pharmaceutical excipient.

In an additional embodiment, the pharmaceutical composition of Form 1, wherein the crystalline form is substantially purified.

An additional embodiment is a method of treating or preventing emesis comprising the administering the composition of Form 1.

In an additional embodiment, the emesis is related to or resulted from chemotherapy treatment.

An additional embodiment is a process for preparing the crystalline form of Form 1 comprising precipitating the crystalline form from a solution comprising (5R,8S)-8-[[(1R)-1-[3,5bis(trifluoromethyl)phenyl]ethoxy]methyl]-8-phenyl-1,3,7-triazaspiro[4.5]decane-2,4-dione and a solvent.

An additional embodiment of the process, wherein the solvent is selected from the group consisting of C₁-C₄ alkyl alcohols, water and mixtures thereof.

An additional embodiment of the process, wherein the precipitation was induced by the sequential addition of anhydrous EtOH and water.

An additional embodiment of the process, wherein
a) the acid is added to the solution at a temperature between about 20° C. and about 40° C.;
b) the water is added at the temperature of about 35° C. to about 45° C., preferably about 40° C.; and
c) the resulting mixture is stirred for two hours and cooled to about 15° C. to about 25° C., preferably 20° C.

### Examples

Form 1 of (5R,8S)-8-[[(1R)-1-[3,5-bis(trifluoromethyl)phenyl]ethoxy]methyl]-8-phenyl-1,3,7-triazaspiro[4.5]decane-2,4-dione was formed from the reaction scheme below.

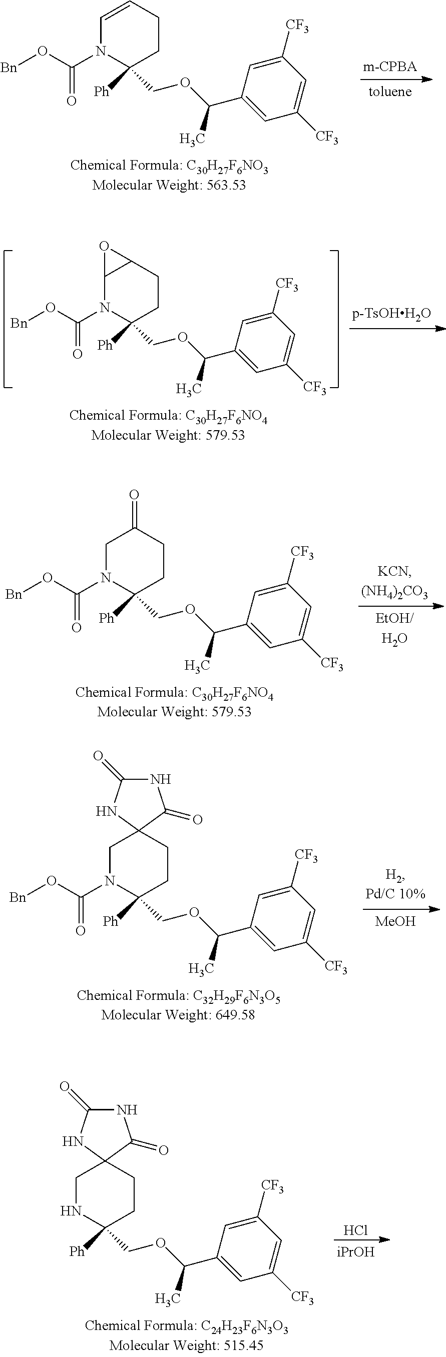

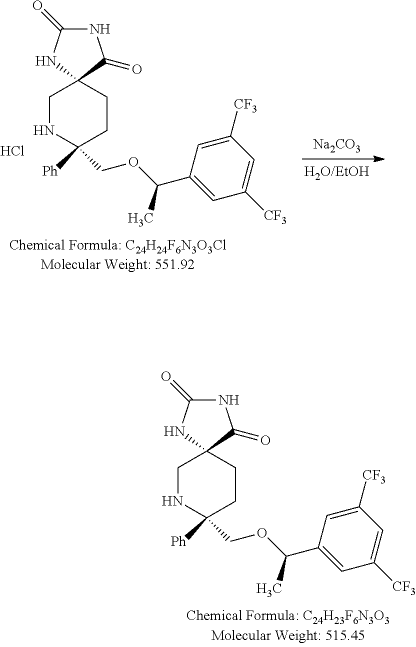

Samples of Forms 1, 2, and 3 were prepared as follows.

Form 1 Crystallization

MK-7035 has been crystallized in the last synthetic step as hydrochloride salt. 15.4 kg of MK-7035 hydrochloride salt was converted to the free base via reaction with sodium carbonate at pH between 9.5 and 12. Phase separation occurred because of this reaction. The organic layer of isopropyl acetate solution was concentrated. EtOH was added, the solution was concentrated again, and water was added as an antisolvent. The resulting solid was filtered, washed with 1:2 (vol:vol) 2-propanol:water mixture and dried under nitrogen for several hours.

The resulting dry cake was stirred with a mixture of 60 kg 2-propanol and 121 kg water at 40° C. for 8 hours, followed by addition of 121 kg water over 1 hour. The mixture was cooled to 20° C. and stirred for 2 hours. The solid was dried under nitrogen and vacuum for several hours. The weight of the dry solid crystals of Form 1 was 10.9 kg.

Forms 2 and 3 Crystallization

Form 2 has been obtained by desolvation of toluene solvate. The toluene solvate was obtained by slurring crystals of Form 1 in toluene at 25 and 50° C. for 24 hours. The resulting solids were filtered and dried by heating to 160° C. in the pan of the thermogravimetric analyzer.

Form 3 has been obtained by desolvation of dichloromethane solvate. The dichloromethane solvate was obtained by slurring crystals of Form 1 in dichloromethane at 25° C. The resulting solid was filtered and dried by heating it to 145° C. in the pan of the thermogravimetric analyzer.

Crystallization of MK-7035 form 2-propanol/water produced Form 1 even when Form 2 and Form 3 seeds were used. The crystallization procedure is described as follows:

100 mg of Form 1 was dispensed in each of two vials. 1.3 mL 2-propanol was added to each vial and the samples were stirred at 60° C. for 2 hours. At the end of the 2-hour period the temperature was set to 40° C. 5 mg of Form 2 and 5 mg of Form 3 were placed separately in 2 vials and 0.8 mL water was added to each vial. The solids did not disperse, the solids stayed in a lump on the water surface. Approximately 0.2 mL of the water along with some solids of Form 2 was withdrawn from the vial using a pipette and was added to one of the vials containing 2-propanol/water solution of MK-7035. The rest of the slurry of Form 2 seeds in water was added in 0.2 mL increments. The same procedure was repeated for the aqueous suspension of Form 3 and the second vial of MK-7035 solution in 2-propanol/water. The seeds of Form 2 and Form 3 did not dissolve. Crystallization did not start for 5 to 10 minutes. The hotplate was turned off. After 15 minutes thick slurry formed in the vial seeded with Form 3, some solids precipitated in the vial seeded with Form 2.

Both samples were aged overnight, and the solids were analyzed by XRPD. The XRPD patterns of the solids from the two slurries corresponded to Form 1, indicating that Form 1 preferentially crystallizes from 2-propanol/water.

X-Ray Powder Diffraction (XRPD)

X-ray powder diffraction studies are widely used to characterize molecular structures, crystallinity, and polymorphism. The X-ray powder diffraction pattern of Form 1 was generated on Bruker D8 Advance Diffraction System. A PW3373/00 ceramic Cu LEF X-ray tube K-Alpha radiation was used as the source.

Solid State NMR

MK-7035 Form 1 was characterized based on its carbon-13 solid-state nuclear magnetic resonance (NMR) spectrum. The carbon-13 spectrum was recorded on a Bruker AVANCE III NMR spectrometer operating at 500.13 MHz, using a Bruker 4 mm H/X/Y triple resonance CPMAS probe. The spectrum was collected utilizing proton/carbon-13 variable-amplitude cross-polarization (VACP) at 83.3 kHz, with a contact time of 3 ms. Other experimental parameters used for data acquisition were a proton 90-degree pulse of 100 kHz, high-power proton TPPM decoupling at 100 kHz, a pulse delay of 10 s, a dwell time of 5.0 μs, an acquisition time of 20.48 ms, and signal averaging for 256 scans. A magic-angle spinning (MAS) rate of 13 kHz was used for data collection. A Lorentzian line broadening of 30 Hz and zero filling to 32768 points were applied to the spectrum before Fourier Transformation. Chemical shifts are reported on the TMS scale using the carbonyl carbon of glycine (176.70 ppm) as a secondary reference.

Differential Scanning Calorimetry (DSC)

DSC data were acquired using TA Instruments DSC Q2000 or equivalent instrumentation. A sample with a weight between 1 and 6 mg was weighed into an open pan. This pan was placed in the sample position in the calorimeter cell. An empty pan was placed in the reference position. The calorimeter cell was closed, and a flow of nitrogen was passed through the cell. The heating program was set to heat the sample at a heating rate of 10° C./min to a temperature of approximately 250° C. When the run was completed, the data were analyzed using the DSC analysis program in the system software. The observed endo- and exotherms were integrated between baseline temperature points that were above and below the temperature range over which the endotherm was observed. The data reported were the onset temperature, peak temperature and enthalpy.

Physical Characterization of MK-7035 Crystalline Form 1

FIG. 1 shows the X-ray powder diffraction pattern of MK-7035 Form 1. Form 1 exhibited characteristic diffraction peaks corresponding to d-spacings of 22.6, 5.2, and 3.2 angstroms. Form 1 was further characterized by the d-spacings of 11.3, 6.2, and 4.3 angstroms.

TABLE 1
Characteristic Peak Position and Corresponding d-Spacing for Form 1

| Peak Position | d-Spacing |
|---|---|
| [°2θ] | [Å] |
| 2.1 | 41.4 |
| 3.9 | 22.6 |
| 6.7 | 13.2 |
| 7.8 | 11.3 |
| 10.4 | 8.5 |
| 13.6 | 6.5 |
| 14.2 | 6.2 |
| 15.2 | 5.8 |
| 16.2 | 5.5 |
| 16.7 | 5.3 |
| 17.2 | 5.2 |
| 18.1 | 4.9 |
| 18.5 | 4.8 |
| 18.9 | 4.7 |
| 19.7 | 4.5 |
| 20.5 | 4.3 |
| 20.9 | 4.3 |
| 21.3 | 4.2 |
| 21.6 | 4.1 |
| 22.7 | 3.9 |
| 23.0 | 3.9 |
| 23.4 | 3.8 |
| 23.7 | 3.8 |
| 24.0 | 3.7 |
| 24.4 | 3.7 |
| 24.7 | 3.6 |
| 26.0 | 3.4 |
| 26.6 | 3.4 |
| 27.8 | 3.2 |
| 28.1 | 3.2 |
| 28.6 | 3.1 |
| 29.2 | 3.1 |
| 29.5 | 3.0 |
| 30.0 | 3.0 |
| 30.5 | 2.9 |
| 31.0 | 2.9 |
| 31.5 | 2.8 |
| 32.1 | 2.8 |
| 32.6 | 2.7 |
| 33.0 | 2.7 |
| 33.6 | 2.7 |
| 34.0 | 2.6 |
| 35.0 | 2.6 |
| 35.4 | 2.5 |
| 36.0 | 2.5 |
| 36.8 | 2.4 |
| 37.3 | 2.4 |

FIG. 2 shows the carbon-13 cross-polarization magic-angle spinning (CPMAS) nuclear magnetic resonance (NMR) spectrum of Form 1. Characteristic peaks are observed at 181.08, 158.94, 145.40, 141.48, 132.60, 131.72, 129.96, 128.58, 126.36, 122.68, 82.09, 81.66, 80.30, 62.96, 60.49, 50.62, 26.75, 24.97, 24.37, 22.60, and 21.65 ppm.

FIG. 3 is a typical DSC curve of the crystalline Form 1. The DSC curve is characterized by a melting endotherm of Form 1 with an extrapolated onset temperature of 203.6° C., a peak temperature of 206.3° C. and enthalpy of 61.9 J/g, followed by an endotherm with an extrapolated onset temperature of 209.8° C., a peak temperature of 210.3° C. and enthalpy of 7.5 J/g, which is due to the melting of Form 2 recrystallized from the melt of Form 1. The second endotherm with an extrapolated onset temperature of 209.8° C., a peak temperature of 210.3° C. and enthalpy of 7.5 J/g may or may not exist depending on the heating rate used to heat the sample. FIG. 3 data were obtained by heating the sample at 10° C./min. With a slower heating rate, including a rate of 10° C./min, there was time during the melting for Form 2 to recrystallize from the melting of Form 1. With a faster heating rate, the sample does not have enough time for Form 2 to recrystallized from the melting of Form 1.

Physical Characterization of MK-7035 Crystalline Form 2 and Form 3.

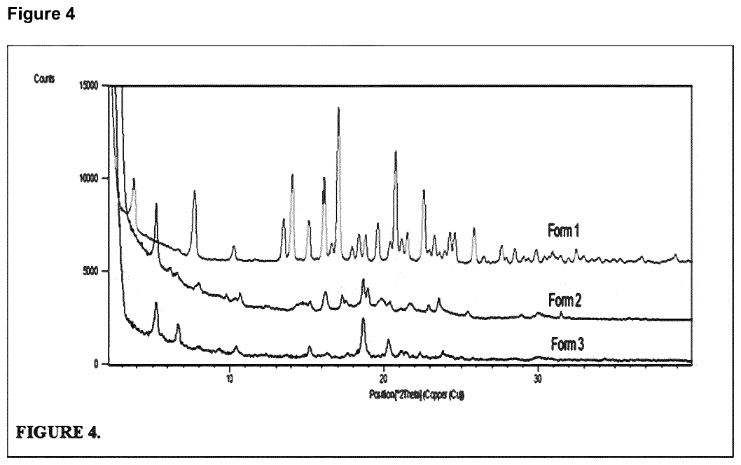

FIG. 4 shows an overlay of the X-ray powder diffraction patterns of MK-7035 Form 1, Form 2, and Form 3.

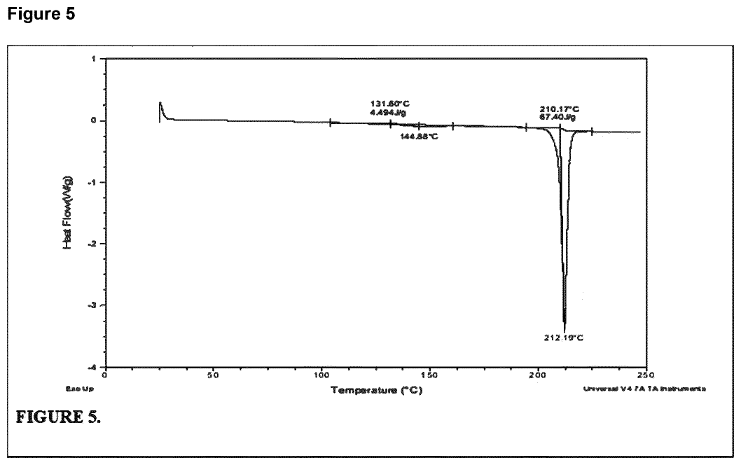

FIG. 5 is a typical DSC curve of the crystalline Form 2. The DSC curve is characterized by a melting endotherm of Form 2 with an extrapolated onset temperature of 210.2° C., a peak temperature of 212.2° C. and enthalpy of 67.4 J/g. The endotherm with an extrapolated onset temperature of 131.6° C., a peak temperature of 144.9° C. and enthalpy of 4.5 J/g, which is due to removal of residual crystallization solvent.

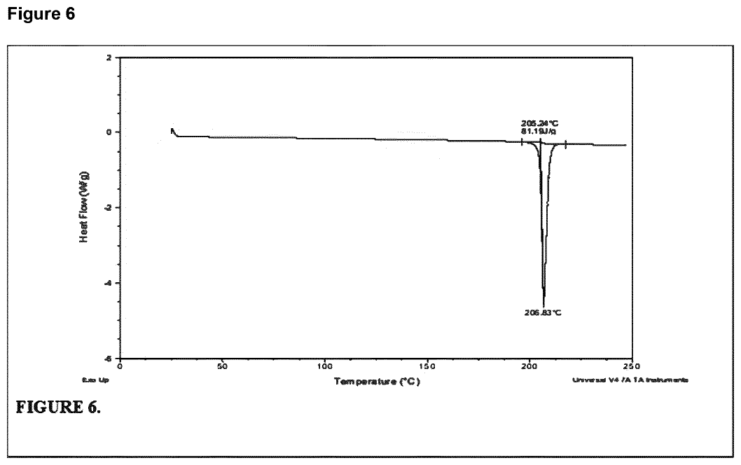

FIG. 6 is a typical DSC curve of the crystalline Form 3. The DSC curve is characterized by a melting endotherm of Form 3 with an extrapolated onset temperature of 205.2° C., a peak temperature of 206.8° C. and enthalpy of 81.2 J/g.

1. A crystalline form of (5R,8S)-8-[[(1R)-1-[3,5bis(trifluoromethyl)phenyl]ethoxy]methyl]-8-phenyl-1,3,7-triazaspiro[4.5]decane-2,4-dione having at least one of the following characteristics:
an X-ray power diffraction (XRPD) spectrum having at least one peak selected from the group consisting of 3.9 (±0.2), 17.2 (±0.2), and 27.8 (±0.2) degrees 2Θ;
a carbon-13 cross polarization magic-angle spinning (CPMAS) nuclear magnetic resonance (NMR) spectrum having at least one peak selected from the group consisting of 181.08, 158.94, 145.40, 141.48, 132.60, 131.72, 129.96, 128.58, 126.36, 122.68, 82.09, 81.66, 80.30, 62.96, 60.49, 50.62, 26.75, 24.97, 24.37, 22.60, and 21.65 ppm; or
a differential scanning calorimetry (DSC) thermogram comprising an endothermic peak at about 206° C.

2. The crystalline form of claim 1 wherein the differential scanning calorimetry (DSC) thermogram further comprises a second endothermic peak at about 210° C.

3. The crystalline form of claim 1, having an X-ray powder diffraction (XRPD) spectrum substantially as shown in FIG. 1.

4. The crystalline form of claim 1, having a carbon-13 cross-polarization magic-angle spinning (CPMAS) nuclear magnetic resonance (NMR) spectrum substantially as shown in FIG. 2.

5. The crystalline form of claim 1, having a differential scanning calorimetry (DSC) thermogram substantially as shown in FIG. 3.

6. A crystalline form of (5R,8S)-8-[[(1R)-1-[3,5bis(trifluoromethyl)phenyl]ethoxy]methyl]-8-phenyl-1,3,7-triazaspiro[4.5]decane-2,4-dione having a carbon-13 cross-polarization magic-angle spinning (CPMAS) nuclear magnetic resonance (NMR) spectrum having at least five peaks selected from the group consisting of 181.08, 158.94, 145.40, 141.48, 132.60, 131.72, 129.96, 128.58, 126.36, 122.68, 82.09, 81.66, 80.30, 62.96, 60.49, 50.62, 26.75, 24.97, 24.37, 22.60, and 21.65 ppm.

7. The crystalline form of claim 1, wherein the crystalline form is thermodynamically stable at temperatures below about 75° C.

8. A pharmaceutical composition comprising the crystalline form of claim 1 and a pharmaceutical excipient.

9. The pharmaceutical composition of claim 8, wherein the crystalline form is substantially purified.

10. A method of treating or preventing emesis comprising administering the composition of claim 8.

11. The method of claim 10, wherein the emesis is related to or resulted from chemotherapy treatment.

12. A process for preparing the crystalline form of claim 1 comprising precipitating the crystalline form from a solution comprising (5R,8S)-8-[[(1R)-1-[3,5bis(trifluoromethyl)phenyl]ethoxy]methyl]-8-phenyl-1,3,7-triazaspiro[4.5]decane-2,4-dione and a solvent.

13. The process of claim 12, wherein the solvent is selected from the group consisting of C₁-C₄ alkyl alcohols, water and mixtures thereof.

14. The process of claim 12, where in the precipitation was induced by the sequential addition of anhydrous EtOH and water.

15. The process of claim 14, wherein
a) the acid is added to the solution at a temperature between about 20° C. and about 40° C.;
b) the water is added at the temperature of about 35° C. to about 45° C.; and
c) the resulting mixture is stirred for two hours and cooled to about 15° C. to about 25° C.

16. The process of claim 15, wherein the temperature of step b) is 40° C.

17. The process of claim 15, wherein the temperature of step c) is 20° C.
